(19) United States
(12) Patent Application Publication
(10) Pub. No.: US 2010/0316761 A1
(43) Pub. Date: Dec. 16, 2010

(54) Nutritional Supplement for Simultaneous Saccharification and Fermentation Medium for the Manufacture of Ethanol

(76) Inventors: Jean-Luc Baret, Veneux Les Sablons (FR); Pierre Labeille, Blainville Sur Orne (FR)

Correspondence Address: JACOBSON HOLMAN PLLC, 400 SEVENTH STREET N.W., SUITE 600, WASHINGTON, DC, 20004, US

(21) Appl. No.: 12/225,245

(22) PCT Filed: Mar. 16, 2007
(86) PCT No.: PCT/FR2007/050937

§ 371 Date: Mar. 24, 2010

(51) Int. Cl. C12G 3/00; A23L 1/28; A23L 1/302; A23L 1/00; A23L 1/30; C12N 1/14

(52) U.S. Cl. 426/11; 426/61; 426/72; 426/618; 426/648; 435/255.1

(57) ABSTRACT

Nutritional supplement for alcoholic fermentation medium based on carbohydrate starting materials, in particular starchy starting materials, comprising, and preferably consisting of, an active ingredient derived from a fermentation with a mould. It can be in liquid or solid form.

The invention relates to a nutritional supplement promoting the industrial production of ethanol by alcoholic fermentation based in particular on a simultaneous saccharification-fermentation process, from a carbohydrate raw material fermentable to ethanol, optionally after saccharification, such as a starchy raw material, in particular from cereals, in particular wheat, and coproducts (brewers' grain, bran). The invention also relates to brewers' grain derived from this process and to a process for the manufacture of yeast under aerobic conditions.

It is known to produce ethanol from a starchy raw material by an enzymatic process comprising a step of liquefying starch with an alpha-amylase intended to solubilize and hydrolyze the starch to dextrins. This step is conventionally followed by a saccharification step with a glucoamylase, also called amyloglucosidase, intended to hydrolyze the dextrins to glucose. The glucose content at the end of saccharification is then substantially 100% of the glucose equivalent corresponding to the starch of said starchy material. In other words, all the glucose potential of the starchy material used was converted to glucose.

The glucose is then fermented during a fermentation step during which it is converted to ethanol by a yeast under anaerobic conditions.

One area of research for improving this type of enzymatic process has consisted in trying to combine the fermentation and saccharification steps. Researchers have tried to carry out the fermentation and the saccharification simultaneously during a single step called simultaneous saccharification and fermentation or SSF. The glucose content of the medium at the start of this single step, that is to say after liquefaction, is then typically less than 3% of the glucose equivalent corresponding to the starch of said starchy material.

Trials have also been carried out in order to limit the duration of the saccharification step such that the glucose content after this step is less than 100% of the glucose equivalent corresponding to the starch of said starchy material. The saccharification step is then termed “presaccharification” or “partial saccharification”.

As will be seen in greater detail in the remainder of this description, the processes for the manufacture of ethanol comprising an incomplete saccharification step, or even no saccharification step, called hereinafter “incomplete saccharification processes”, involve specific constraints. Technical solutions adopted in the context of conventional processes with complete saccharification before fermentation cannot therefore a priori be used with these processes.

In the context of the incomplete saccharification processes, the expression “simultaneous saccharification and fermentation medium” refers to a medium where the fermentation and at least part of the saccharification occur.

There is a permanent need for improving the ethanol yield of the processes for the production of ethanol, in particular based on incomplete saccharification.

The object of the present invention is to respond to this need.

According to the invention, this aim is achieved by means of a nutritional supplement for a simultaneous saccharification and fermentation medium in a process for the manufacture of ethanol by fermentation from a starchy raw material containing and preferably consisting of, an active ingredient derived from a fermentation with a mold. It may be in liquid or solid form.

As will be seen in greater detail in the remainder of the description, the presence of such a nutritional supplement in a simultaneous saccharification and fermentation medium has proved particularly advantageous for the growth and the efficiency of the yeast. This has resulted in a significant improvement in the yield of the process for the manufacture of ethanol from a starchy raw material.

Preferably, the process according to the invention comprises one, and preferably still several, of the following optional characteristics:
The nutritional supplement is a solid or liquid mixture of said active ingredient and at least one useful enzyme. The notion of useful enzyme includes in particular one or more enzymes having an amylase, in particular glucoamylase, proteolytic or xylanase activity, and combinations thereof.
The active ingredient present in the nutritional supplement according to the invention is in crude form, that is to say that it comprises the substrate used during the fermentation with said mold, or in extracted form, that is to say that it is isolated from said substrate. It is optionally mixed with additional enzymes. Preferably, the substrate is wheat bran.
The active ingredient is chosen from the group consisting of ergosterol, N-acetylglucosamine, vitamins, in particular vitamin B, nucleic acids, amino acids and, preferably, mixtures thereof. Among the amino acids, one or more, preferably all, of the following amino acids are desired: alanine, arginine, asparagine, aspartic acid, valine, leucine, glutamic acid. Among the vitamins, the presence of various B vitamins, for example B1, B2, B3, B6 and inositol, vitamin E, among others is noted.
The nutritional supplement has, during its introduction into the simultaneous saccharification and fermentation medium, in particular because of an appropriate choice of said mold, the following enzymatic activities:
   glucoamylase: at least 500 GU, preferably 750 GU, preferably still 1500 GU per gram of dry matter, and/or preferably,
   proteolytic: at least 100 PU, preferably at least 400 PU per gram of dry matter, and/or, preferably,
   xylanase: at least 100 XU, preferably at least 400 XU per gram of dry matter.
The nutritional supplement is derived from a fermentation, preferably in solid medium of a substrate, preferably wheat bran, with a mold, in particular chosen from the Aspergillus niger strains, preferably chosen from the strains ATCC 201202, ATCC 76060, ATCC 76061, MUCL 28815, MUCL 28816, NRRL 3112 or from the Aspergillus orizae strains, preferably chosen from the strains ATCC 22788 and ATCC 42149.
The nutritional supplement is obtained by means of a process according to the invention comprising the following steps:
   i) taking wheat bran,
   ii) moistening, acidifying to a pH of between 4 and 5, preferably with nitric acid (HNO₃) and heat treating said bran so as to pasteurize it or sterilize it, the heat treatment being preferably after the moistening,
   iii) inoculating the resulting wheat bran with an Aspergillus niger strain, chosen from ATCC 201202, ATCC 76060, ATCC 76061, MUCL 28815, MUCL 28816, NRRL 3112, or with an Aspergillus orizae strain chosen from ATCC 22788 and ATCC 42149,
   iv) fermenting the wheat bran in the solid state in a reactor which is periodically stirred, at a temperature of 28° C. to 38° C., the moisture content being maintained between 45% and 65%, preferably between 50% and 60% by weight, under aeration conditions designed to avoid a permanent accumulation of carbon dioxide in the wheat bran until the fermentation product has the following minimum enzyme activity values:
      glucoamylase: at least 500 GU, preferably 750 GU, preferably still 1500 GU per gram of dry matter and, preferably,
      proteolytic: at least 100 PU, preferably 400 PU per gram of dry matter,
      xylanase: at least 100 XU, preferably 300 XU per gram of dry matter.
The nutritional supplement is an enzymatic complex obtained according to a process described in US 2002 037342.

The invention also relates to a process for the manufacture of ethanol from a carbohydrate, in particular starchy, material comprising, after a liquefaction step, a saccharification step and then a fermentation step, or after a liquefaction step and optionally a presaccharification step, a simultaneous saccharification and fermentation step in a simultaneous saccharification and fermentation medium whose glucose content at the start of the simultaneous saccharification and fermentation step is at most equal to 95% of the glucose equivalent corresponding to the carbohydrate material, in particular to the starch of said starchy material. The process according to the invention is remarkable in that a nutritional supplement according to the invention is introduced into the fermentation or simultaneous saccharification and fermentation medium. This introduction may be carried out directly into the fermentation or simultaneous saccharification and fermentation medium, or during a step upstream.

Surprisingly, the presence, in the fermentation or simultaneous saccharification and fermentation medium, of a nutritional supplement according to the invention, and in particular of a wheat bran fermented with a mold, has proved particularly favorable for increasing the efficiency of the yeast. The latter then converts a larger quantity of glucose to ethanol with a more favorable kinetics.

Preferably, the process according to the invention comprises one, and preferably still several, of the following optional characteristics:
The glucose content at the start of the simultaneous saccharification and fermentation step is at most equal to 3%, preferably to 2%, preferably still at most equal to 1%, of the glucose equivalent corresponding to the starch of said starchy material. In other words, the process does not comprise a saccharification step or a presaccharification step.
As the nutritional supplement is introduced in crude form, the quantity of said nutritional supplement is greater than or equal to 4 kg of dry matter, preferably to 14 kg of dry matter and/or less than or equal to 60 kg of dry matter, preferably to 34 kg of dry matter, preferably still less than 19 kg of dry matter per ton of starch, preferably about 17 kg of dry matter per ton of starch.
The nutritional supplement is prepared by fermentation of a substrate identical to the carbohydrate material to which the process for the manufacture of ethanol is applied.
The simultaneous saccharification and fermentation step is entirely carried out in the absence of an air or oxygen supply (no aerobic phase strictly speaking).
The simultaneous saccharification and fermentation step comprises an initial aerobic phase.
A yeast is used as ethanolic fermentation agent, in particular a yeast of the genus Saccharomyces, in particular Saccharomyces cerevisiae. More preferably, the yeast is functional at ethanol concentrations greater than 95 g/l.
During the process, said nutritional supplement constitutes the main nutritional source for the yeast and/or the main source of ergosterol and/or the main source of nitrogen and/or of amino acids and/or of phosphorus and/or of sulfur and/or of vitamins. Preferably, it constitutes the sole source of ergosterol and/or of nitrogen and/or of amino acids and/or of phosphorus and/or of sulfur and/or of vitamins.

The process according to the invention further makes it possible to manufacture brewers' grain of remarkable nutritional quality. The invention therefore further relates to the brewers' grain derived from a process for the manufacture of ethanol according to the invention and containing more than 5 g, preferably more than 20 g and/or less than 100 g, preferably less than 35 g, preferably still about 30 g of ergosterol per ton of dry matter of brewers' grain.

Preferably, said brewers' grain further contains more than 40%, preferably more than 50% of crude proteins, as percentages on the basis of the dry matter. As will be seen in the remainder of the description, such protein contents are made possible by the consumption of part of the hemicelluloses and of the fibers during the prefermentation or the simultaneous saccharification and fermentation.

The invention finally relates, in general, to the use of a nutritional supplement according to the invention for promoting an alcoholic fermentation intended for the manufacture of ethanol or a prefermentation intended for the manufacture of a yeast.

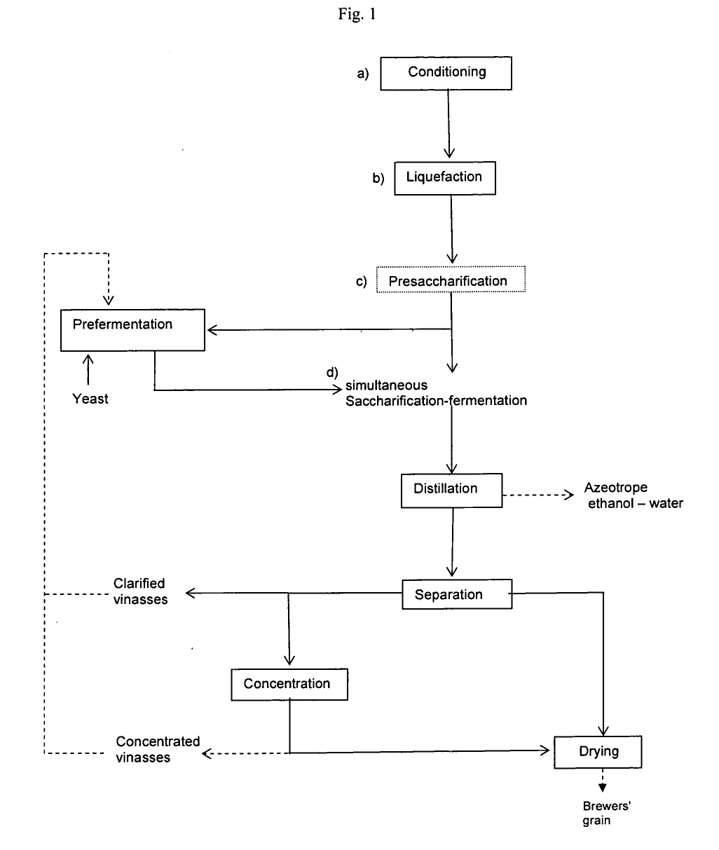

Other characteristics and advantages of the present invention will emerge further in the light of the description which follows and upon examining the appended drawing where FIG. 1 represents a diagram of part of a process for the manufacture of ethanol according to the invention.

Processes for the manufacture of ethanol by fermentation of starchy material are well known to the person skilled in the art, and could be adapted to conform to the invention. Persons skilled in the art are therefore able to clarify, where appropriate, certain points of the following description.

The process described below relates to the manufacture of ethanol from wheat, but can apply to any starchy material, in particular to any cereal extract. The embodiments described below are therefore only examples and it would be possible to modify them, in particular by substitution of technical equivalents, without departing from the scope of the invention.

A process for the manufacture of ethanol from wheat according to the invention may, by way of example, comprise the following steps:
a) a step of conditioning wheat so as to prepare ground wheat starch,
b) a step of liquefying the starch, in particular in the presence of an alpha-amylase, so as to hydrolyze the starch to dextrins,
c) optionally a presaccharification step, in particular with a combination of enzymes, so as to hydrolyze the dextrins to fermentable sugars (glucose, maltose, maltotriose) and the non-starch constituents, and
d) a simultaneous saccharification and fermentation step, in a simultaneous saccharification and fermentation medium containing the dextrins and/or said fermentable sugars and a yeast, so as to produce ethanol.

These various steps are represented in FIG. 1.

Step a) consists in preparing ground wheat starch, for example flour, by conditioning said wheat.

The wheat flour is then mixed in a mixer with water, optionally vinasse, acid and a liquefaction enzyme, so as to form a “mashed wort”.

The mashed wort typically contains from 25 to 35% by mass of dry matter, preferably from 30 to 35%. The percentage of dry matter is determined so as to limit the energy expenditure while preserving a satisfactory fluidity. For economic considerations, the percentage of dry matter is as high as possible in order to limit the costs of evaporation of the vinasses in the remainder of the process. The quantity of flour supplied to the mixer is regulated by a metering device, or by a weighing belt.

Depending on the liquefaction enzyme used, the pH should be adjusted with an acid solution, e.g. sulfuric acid. Depending on the enzymes used, conventionally in particular thermostable bacterial alpha-amylases, a calcium salt may be optionally used. The acid flow rate is regulated by means of a pH probe fitted onto the water/vinasse mixture before the masher. The pH can conventionally vary between 5 and 6.5, depending on the enzymes used.

The liquefaction (step b)) is then carried out at a temperature of between 80° C. and 95° C. The mashed wort may be heated to this temperature with the aid of a direct injection of steam into the liquefaction tank by pipes or by a jet cooker. In the second case, the mashed wort is heated for a few seconds at a temperature of between 100° C. and 150° C. by means of a steam injection into a nozzle before being rapidly cooled to between 80° C. and 95° C. The liquefaction tanks may be stirred.

The preferred characteristics of the liquefaction step are the following:
temperature: 80° C. to 90° C.
pH: 5.5-6.5
dry matter: 30% to 35%
residence time: 30 minutes to 2 hours.

Step b) leads to the hydrolysis of the starch to dextrins.

In the case of presaccharification (step c)), the liquefied wort is cooled in heat exchangers of the plate exchanger or tubular exchanger type at a temperature of between 50° C. and 60° C.

In some cases, the liquefied wort may be diluted with a diluent such as water or recycled vinasses or the phlegmas coming from the distillery.

In step c), an optional step, the liquefied wort is preferably brought into contact with an enzymatic complex having the desired enzymatic activities.

The flow rates of the enzymes are preferably controlled by the flow rate of the incoming wort, by the concentration of glucose produced and by the content of starch remaining. The objective of this control is to prepare a solution free of starch at the end of fermentation.

The preferred characteristics of step c) of presaccharification are the following:
temperature: 50° to 60° C.
pH: 4 to 5
residence time: until the desired glucose content is obtained, generally less than 24 h.

The presaccharification tanks are subjected to mechanical stirring, allowing good homogenization of the wort during saccharification and thereby a facilitated contact between the enzymes and the dextrins to be hydrolyzed.

The use of the enzymatic complex derived from the fermentation of wheat bran by a mold advantageously makes it possible to reduce the viscosity of the saccharified wort and to increase the concentration of nitrogen in said wort.

According to the invention, the saccharification is not pursued up to complete hydrolysis of the dextrins to glucose, but interrupted while the hydrolysis is only partial. According to the invention, the glucose level at the end of the presaccharification step is less than 95%, preferably less than 50%, preferably still less than 5%. The hydrolysis therefore continues during the simultaneous saccharification and fermentation step.

If the process according to the invention does not comprise a partial saccharification, or “presaccharification”, step, the glucose content at the start of step d) of simultaneous saccharification and fermentation is at most equal to 3%, preferably to 2%, preferably still at most equal to 1%, of the glucose equivalent corresponding to the starch of said starchy material.

In step d), the wort is brought into contact with a yeast in the simultaneous saccharification and fermentation medium.

It is also brought into contact with a nutritional supplement according to the invention. The order of incorporation of the yeast and of the supplement is not important.

The whole is stirred during the entire duration of step d).

All the yeasts used for the production of ethanol may be used, in particular yeasts of the genus Saccharomyces. Usually, the yeast is introduced into the simultaneous saccharification and fermentation medium under aerobic conditions. During this phase, the yeast undergoes a number of cell divisions. The yeast does not convert glucose to ethanol in this case, but on the contrary consumes glucose for its growth. To improve this cell growth, it is known to add nutritional supplements to the simultaneous saccharification and fermentation medium. Moreover, in order to limit cell growth, whose accompanying glucose consumption reduces the yield of ethanol production, it is preferable to inoculate the simultaneous saccharification and fermentation medium with a quantity of yeast such that the production of ethanol by the yeasts rapidly makes it possible to reach ethanol concentrations in the culture medium which inhibit cell growth of the yeast.

According to a first feature, step d) starts under aerobic conditions for a period necessary for sufficient multiplication of the yeast.

The simultaneous saccharification and fermentation medium is then placed under anaerobic conditions. The yeast then converts the glucose to ethanol.

According to a second feature, step d) is entirely performed under anaerobic conditions. The presence of the nutritional supplement according to the invention makes it possible to dispense with the initial aerobic phase.

The preferred characteristics of step d) of simultaneous saccharification and fermentation are the following:
Temperature: from 30° C. to 35° C.
pH: adjusted at the start of step d) with acid (e.g. sulfuric acid) to between about 3.5 and about 5, preferably between about 3.8 and about 5, preferably still between about 4 and about 5, better still between about 4 and about 4.5.
After adjusting the pH at the start of step d), by virtue of the buffer effect of the nutritional supplement, no provision is made for the regulation of the pH during the remainder of step d).
Yeast inoculum: about 10⁶ to about 5×10⁸ CFU (colony forming units) of yeast per ml of simultaneous saccharification and fermentation medium, preferably about 10⁷ CFU of yeast per ml of simultaneous saccharification and fermentation medium.
20% to 35%, in particular 20% to 30% of DM.
Residence time: 20 hours to 72 hours, in particular 20 hours to 60 hours. The residence time increases with the DM.

The simultaneous saccharification and fermentation medium according to the invention contains a nutritional supplement according to the invention. This supplement may be introduced directly into the simultaneous saccharification and fermentation medium or during a step upstream. Surprisingly, the addition to the simultaneous saccharification and fermentation medium of such a nutritional supplement in particular of a wheat bran fermented with a mold, proved particularly favorable for increasing the efficiency of the yeast. The latter then converts a larger quantity of glucose to ethanol with a more favorable kinetics. In addition, the nutritional supplement according to the invention makes it possible to reduce the duration of the aerobic phase at the start of the simultaneous saccharification and fermentation step, or even to eliminate it. Advantageously, the yield thereof is improved. Advantageously still, the nutritional supplement according to the invention makes it possible to reduce the cell mortality compared with an identical culture performed in the absence of said nutritional supplement. Finally, its presence has a buffer effect, avoiding having to regulate the pH.

According to the invention, preferably, the simultaneous saccharification and fermentation medium initially contains, per 1000 kg of starch initially introduced, between 2.5 kg and 35 kg of nutritional supplement, in particular fermented wheat bran, in particular between 8 and 10 kg of nutritional supplement, in particular fermented wheat bran, per 1000 kg of starch.

The presence in the simultaneous saccharification and fermentation medium of fermented wheat bran therefore makes it possible to considerably reduce the simultaneous saccharification and fermentation time.

It is thought that this nutritional supplement provides nutrients which are perfectly suitable for the yeast, in optimum proportions and in an optimum form, in particular for the anaerobic cultures.

Without being bound by a theory, the inventors explain these results in the following manner. They discovered the presence, in the nutritional supplement used according to the invention, of amino acids necessary for the yeast. These amino acids are thus thought to contribute to the nitrogen nutrition of the yeast in a very efficient manner, in particular much more efficient than the simple ammonium salts used up until now, additionally reducing the synthesis of fermented coproducts such as glycerol and thereby improving the ethanol yield.

The presence of vitamins in the nutritional supplement according to the invention is also thought to make it possible to explain a better efficiency of the yeast.

Finally, the inventors have discovered that the nutritional supplement according to the invention contains ergosterol and N-acetylglucosamine, which are important constituents of the yeast. Their presence in the simultaneous saccharification and fermentation medium helps the growth and the correct functioning of the yeast under complete anaerobic conditions. Under these conditions, its synthesis by the yeast is indeed impossible. The presence of ergosterol therefore makes it possible advantageously to reduce, or even eliminate the aerobic phase at the start of the simultaneous saccharification and fermentation.

The trials of table 1 below have nevertheless demonstrated that the addition of the nutrients identified does not lead to a simultaneous saccharification and fermentation medium as efficient as the addition of a nutritional supplement according to the invention. It is therefore indeed the combination of all of the constituents of this nutritional supplement, in the proportions resulting from the fermentation with a mold, and preferably their presentation in the form of a wheat bran which are responsible for the exceptional performances obtained.

It is possible for the nutritional supplement not to have particular enzymatic activities or optimum enzymatic activities. Enzymatic additions are then necessary.

Preferably, however, the nutritional supplement according to the invention also has enzymatic activities useful in the context of the process for the manufacture of ethanol. In particular, it is preferable that the nutritional supplement has a glucoamylase activity greater than 500 GU per gram of dry matter. Preferably still, the nutritional supplement has a proteolytic activity greater than 100 PU per gram of dry matter and/or a xylanase activity of at least 100 XU per gram of dry matter.

Thus, although it is possible to envisage the use of other enzymes such as purified enzymes or purified enzyme complexes, it is preferable, according to the invention, to use a nutritional supplement fermented under conditions allowing it to have said enzymatic activities. Preferably, this nutritional supplement is then introduced in step d), and/or where appropriate, in step c), as an enzymatic complex.

Preferably, the nutritional supplement is a fermented wheat bran prepared or capable of being obtained according to the following process according to the invention:
i) taking wheat bran,
ii) moistening and heat-treating said bran so as to pasteurize it or sterilize it, the heat treatment being preferably subsequent to the moistening,
iii) inoculating the resulting wheat bran with an Aspergillus niger strain, chosen from ATCC 201202, ATCC 76060, ATCC 76061, MUCL 28815, MUCL 28816, NRRL 3112, preferably from ATCC 76061 and NRRL 3112, preferably still the strain ATCC 76061 or from the Aspergillus orizae strains ATCC 22788 and ATCC 42149,
iv) fermenting the wheat bran in the solid state, preferably in the form of a layer over 10 cm thick, in a reactor which is periodically stirred, at a temperature of 28° C. to 38° C., the moisture content being maintained between 45% and 65%, preferably between 50% and 60% by weight, under aeration conditions designed to avoid a permanent accumulation of carbon dioxide in the wheat bran until the fermentation product has the following minimum enzymatic activity values:
   glucoamylase: at least 500 GU, preferably 750 GU, preferably still 1500 GU per gram of dry matter and, preferably,
   proteolytic: at least 100 PU, preferably 400 PU per gram of dry matter, and preferably still,
   xylanase: at least 100 XU, preferably 400 XU per gram of dry matter.

In step i), the wheat bran is preferably chosen so as to have a proportion of at least 40% by mass of particles of less than 1 mm.

In step ii), the wheat bran should be moistened and heat treated in order to pasteurize it or sterilize it. It is advantageous that the heat treatment does not precede the moistening because poor fermentation results have been observed where the bran is heat treated before it is moistened.

The heat treatment may consist of heating, for example, in an autoclave. A 20 minute treatment in an autoclave between 120 and 121° C. was found to be very satisfactory. Less severe pasteurization conditions at 105° C., for 15 minutes, in an oven are also suitable. It is also possible to carry out the heat treatment of the bran by injecting steam therein, which makes it possible to simultaneously moisten the bran.

Preferably, the pH is adjusted, preferably with nitric acid, during the moistening in a range of 4 to 5.5 in order to improve the pasteurizing effect of the heat treatment and the onset of the desired fermentation. The use of nitric acid is particularly advantageous, nitric acid being also used as nitrogen source by the mold.

In addition to its sterilizing function, the heat treatment has the effect of promoting the gelatinization of the starch contained in the wheat bran and, therefore the availability of this substrate for the Aspergillus niger and Aspergillus orizae fungi, which allows more efficient fermentation.

The moistening of the bran is important because the water content influences the performance of the fermentation. It is determined such that the water content of the bran is, at the start of step iv) of fermentation, in the range of 50-60%, preferably 50-55%, of the total mass of the bran and of the water.

In step iii), the inoculation of the wheat bran may be carried out with any appropriate inoculum. Persons skilled in the art know many ways of preparing a suitable inoculum from a selected strain. The inoculation dose is advantageously at least 10⁷ spores/gram of initial dry matter.

In step iv), the fermentation may be performed in any appropriate reactor. Examples of reactors which can be used are those described in the article by A. DURAND et al., published in Agro-Food-Industry Hi-Tech (May-June 1997, pages 39-42).

The fermentation should be performed until the glucoamylase activity is at least 500 GU, preferably at least 750 GU, preferably still at least 1500 GU per gram of bran dry matter, that is to say normally for a period of 1 to 3 days, preferably of 30 to 60 hours. Below 1 day, the fermentation is too incomplete. After 3 days, the fermentation is complete or practically complete such that it would be uneconomical to extend it further.

The temperature of the medium is maintained at between 28° C. and 38° C., preferably between 32° C. and 36° C., which corresponds to the optimum activity range known for the strains used. Advantageously, for this purpose, the air temperature is set at between 34° C. and 38° C. for the first few hours of fermentation in order to promote germination of the spores, and then reduced to between 28° C. and 32° C. for the remainder of the fermentation in order to contribute toward regulating the temperature of the medium.

The moisture content of the wheat bran is normally between 50% and 60%. The moisture level may however vary by +/−5% units from the 50-60% interval for a relatively short period between two successive adjustments of moisture level or at the end of fermentation. It is advisable, in any case, not to fall below a moisture level of 45%. The moisture level of the culture medium tends to decrease during the culture through evaporation under the effect of the increase in temperature generated by the fungal growth, said medium being a poor heat conductor. It is therefore necessary to maintain the moisture content during fermentation, for example by periodically supplying water in order to compensate for the loss of water from the medium. The quality of water used also plays a significant role. It is possible to use running water of good quality or distilled water.

The pH of the fermentation medium is not usually regulated. As explained above, it is preferably initially adjusted to between 4 and 5.

If its starting value is close to 6.0-6.4, the pH decreases to 3.8-4.2, during the culture, and then rises at the end. This rise is generally correlated with the sporulation phase for the fungus. Monitoring the pH variation constitutes a good indicator of the state of the culture.

The fermenter should be aerated, preferably continuously, in order to provide the oxygen necessary for the fermentation and to avoid the excessive accumulation of carbon dioxide produced by fermentation. In addition, aeration contributes toward controlling the temperature and the moisture of the culture medium. The air is preferably substantially saturated with water in order to limit the tendency of the medium to become dry. It is difficult to give quantitative indications on the rate of aeration because many variables, in particular the size and the geometry of the reactor, the quantity of the bran loaded, and the like, are involved. Simple routine trials will, however, allow persons skilled in the art to easily determine a suitable aeration rate in each practical case, generally an air flow rate of 1 to 2 m³/h per kg of dry matter is appropriate, the excess pressure is preferably between 0.5 and 1 bar.

The bran load in the fermenter should be periodically stirred, during the fermentation with the aid of stirring means such as stirring arms, blades or spatulas, or endless screws in order to avoid the formation of impenetrable masses and so that the aeration affects the entire mass of bran as homogeneously as possible. Surprisingly, the strains used withstand the stirring. An excessively vigorous stirring should, however, be avoided.

The fermented wheat bran used in the process according to the invention may be dried or frozen for its preservation, if desired, or cooled and used without additional processing.

The drying is preferably carried out at a moderate temperature so as not to affect the enzymatic activity. Heating in an oven at 40° C. was found to be appropriate. At the industrial level, dry air between 35 and 45° C. is preferably ventilated, depending on the mold used. The freezing, for its part, may be carried out on the moist product at low temperature, for example at −20° C.

US 2002 037342 discloses an enzymatic complex with glucoamylase, proteolytic and xylanase activities, obtained by fermentation of wheat bran with Aspergillus strains. This complex may be used as nutritional supplement according to the invention.

In general, it is not possible to predict the effect, in the context of a process comprising a saccharification step which is at least partially simultaneous with the fermentation, of an enzyme, or of a multienzymatic composition whose use is known in the context of a process with separate saccharification and fermentation steps. This is in particular verified when the addition of a composition involves, like the composition of US 2002 037342, the incorporation of substances such as hemicelluloses capable of increasing the viscosity during the fermentation.

This document describes the use of this composition in order to improve the conditions of the saccharification step. It does not at all suggest that it could be of interest with a process not comprising a saccharification step, or comprising only a presaccharification step.

Indeed, the optimum conditions for the saccharification and for the fermentation are very different. In particular, the saccharification is commonly carried out at about 60° C., that is to say at a temperature which is not suitable for the yeasts normally used for the fermentation. Conversely, as illustrated by table 7 of US 2002 037342, the enzymes of the composition of US 2002 037342 have an optimum enzymatic activity at the saccharification temperatures, but, in line with every expectation, are thought to have an impaired enzymatic activity in the case of simultaneous saccharification and fermentation.

In addition, the enzymes of the composition of US 2002 037342 are known to reduce the viscosity of the saccharification medium at the saccharification temperature (see table 8), but persons skilled in the art could expect this decrease in viscosity not to occur at the fermentation temperature, in any case to an extent making the viscosity of the simultaneous saccharification and fermentation medium acceptable.

Persons skilled in the art would therefore not have attempted to use the composition of US 2002 037342 in a process for the manufacture of ethanol which does not comprise a saccharification step, or which comprises only a presaccharification step. This is even less likely since the elimination of the saccharification or a limitation of its duration, can sometimes lead to a simultaneous saccharification and fermentation medium that is contaminated in an unacceptable manner unless large quantities of bacteriostatic agents are added. Indeed, the saccharification temperature allows protection against bacterial contamination.

The invention therefore also relates to the use of a wheat bran obtained according to the process described above or in accordance with the wheat bran described in US 2002 037342 for promoting alcoholic fermentation in a simultaneous saccharification and fermentation medium. Preferably, more than 4 kg of this bran are present in the simultaneous saccharification and fermentation medium at the start of step d) per 1000 kg of starch.

Advantageously, in the preferred embodiment of the invention, the nutritional supplement is a multienzymatic complex obtained by fermentation of a wheat bran with a mold and:
constitutes a means of upgrading the bran derived from step a) of preparing wheat flour,
simultaneously provides several useful enzymes, thus simplifying the process, and
is an excellent nutritional supplement for the yeast.

To the latter effect, the quantity by mass of multienzymatic complex should however be much higher than according to the prior art.

Current research tends to manufacture increasingly concentrated multienzymatic complexes in order to limit the quantities by mass introduced. The processes are thereby simplified.

Contrary to this tendency, the inventors have therefore discovered that by contrast, the incorporation of more than 4 kg of dry matter, and preferably more than 14 kg of dry matter, preferably still of about 19 kg of dry matter of fermented wheat bran per ton of starting starch makes it possible to improve the overall efficiency of the process for the manufacture of ethanol.

At the end of step d), the fermented wort or “wine” derived from the simultaneous saccharification and fermentation, after passing through a heat exchanger, feeds the distillation columns.

The vinasses leaving at the column base are sent to the room for separating the brewers' grain so as to be clarified by conventional methods of the centrifugation type in order to separate the soluble matter from the insoluble matter.

The moist brewers' grain obtained following this separation step is composed of about 35% of dry matter, the clarified vinasses are composed of about 7% to 10% of dry matter.

The clarified vinasses are concentrated by evaporation under vacuum in order to obtain a syrup or “concentrated vinasse” with a dry matter content of close to 35%.

The syrup obtained may then be mixed with the moist brewers' grain. The mixture is then dried and about 350 kg of brewers' grain thus dried are obtained per ton of wheat, the dry matter content of this brewers' grain being about 90%.

The brewers' grain obtained after drying has remarkable nutritional characteristics, in particular for livestock, and is also a subject of the invention. The 350 kg of brewers' grain obtained according to the invention indeed contain between 2 g and 35 g of ergosterol, preferably about 10 g. Now, ergosterol is a precursor for the synthesis of vitamin D2 and is therefore beneficial to health and also has nutritional benefits for the yeasts, in particular under anaerobic conditions.

Furthermore, in the case where the nutritional supplement is a wheat bran fermented according to the process according to the invention described above, the brewers' grain obtained contain a surplus of proteins.

The alcohol vapors of the columns are condensed in heat exchangers. The azeotrope recovered at the top of the column is dried by conventional methods, for example using molecular sieves.

The process according to the invention makes it possible to obtain about 375 liters to 390 liters of ethanol for an initial quantity of 1000 kg of wheat, that is, remarkably, up to 91% of the stoichiometric yield of conversion by fermentation of the glucose to ethanol for a wheat containing about 60% starch.

Without being bound by this theory, the inventors explain these results by the replacement of an exogenous nitrogen supply, for example in the form of ammonium sulfate, with free amino nitrogen. This replacement advantageously reduces the synthesis of fermentative coproducts, in particular of glycerol and therefore increases the yield.

The invention also relates to a process for the manufacture of the yeast under aerobic conditions, or “propagation of yeast” which can be used in particular in prefermentation in order to prepare a yeast used in a fermentation medium or in a simultaneous saccharification and fermentation medium for a process for the alcoholic fermentation of glucose to ethanol.

In this context, the aim of the prefermentation step is generally to increase the concentration of yeast in the prefermentation medium from about 10⁶-10⁷ CFU per ml to a minimum of about 10⁸ CFU per ml of prefermentation medium, preferably a minimum of about 5×10⁸ CFU per ml of prefermentation medium.

The prefermentation is carried out in prefermenters where the temperature is rigorously controlled and regulated, for example by a system of cooling plates in which a cooling fluid circulates inside or outside the prefermentation tanks. Any multiplication of microorganisms indeed causes a rise in temperature which can become inhibitory for the propagation of the yeasts. The temperature in the prefermenters is conventionally maintained between 30° C. and 35° C.

A supply of oxygen, for example in the form of compressed air, is essential for the propagation of the yeast.

In order to promote the development of the yeasts, it is known to add to the nutrient medium:
nitrogen supplied in various forms such as urea, ammonia, ammonium salts,
phosphorus supplied in various forms such as phosphoric acid, phosphates,
sulfur supplied in various forms such as sulfuric acid, sulfates,
essential minerals.

According to the prior art, wort leaving the liquefaction, saccharification or presaccharification step is also added to the nutrient medium. This wort thus provides fermentable sugars but leads to a reduction in the overall yield of the alcoholic fermentation. A need therefore exists for a process for the manufacture of yeast under aerobic conditions which can be used in prefermentation in order to prepare a yeast for use in a fermentation medium or in a simultaneous saccharification and fermentation medium for a process for alcoholic fermentation of glucose to ethanol and which would limit this reduction in yield.

According to the invention, this aim is achieved by adding to the prefermenters at least part of the clarified vinasses, that is to say obtained at the end of the separation step, and/or the concentrated vinasses obtained during the carrying out of a process for the manufacture of ethanol by alcoholic fermentation from carbohydrate raw materials, in particular starchy raw materials and/or according to the invention.

The vinasses may be obtained from a process for the manufacture of ethanol comprising a step of simultaneous saccharification and fermentation or in which the saccharification and fermentation steps are completely separate.

Preferably, the vinasses result from a process for the manufacture of ethanol according to the invention, into which a nutritional supplement according to the invention has been introduced.

Thus, the invention also relates to a process for the manufacture of ethanol as defined above, in which yeasts obtained by a process for the manufacture of yeast as defined above are used for the ethanol fermentation, which process uses vinasses obtained from said process for the manufacture of ethanol.

The use of the vinasses resulting from a process for the manufacture of ethanol according to the invention, into which a nutritional supplement according to the invention has been introduced, for the production of yeast inside the prefermenters, is particularly advantageous since it makes it possible to separate the production of ethanol by the yeasts, on the one hand, and the cell growth of the yeasts, on the other hand. Indeed, the vinasses resulting from a process for the manufacture of ethanol according to the invention constitute a nutrient medium which makes it possible to grow the yeasts up to a level compatible with a high inoculation of the fermentation or simultaneous saccharification and fermentation medium, which causes the yeasts to use the fermentable sugars present in the fermentation or simultaneous saccharification and fermentation medium mainly for the production of ethanol, and not for cell growth, as was indicated above. Thus, the fermentable sugars derived from the starting carbohydrate substrate are used mainly for the production of ethanol, while it is mainly the nonfermentable sugars which serve for cell growth. The overall yield of ethanol production from the starting carbohydrate substrate is thereby improved compared with the processes in which the fermentable sugars are used by the yeasts both for the production of ethanol and for cell growth.

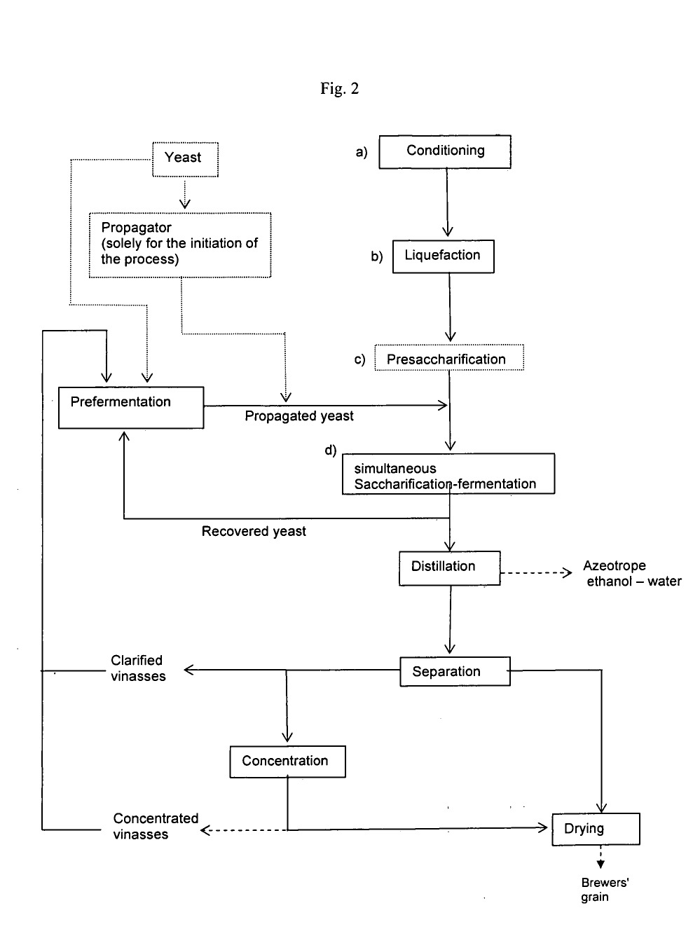

A particular embodiment of such a process is described in FIG. 2.

Preferably, the prefermentation nutrient mixture contains vinasses and is free of intermediate products obtained during the use of a process for the manufacture of ethanol by alcoholic fermentation from starchy raw materials, in particular the vinasses are not mixed with wort derived from the intermediate steps of a process for the manufacture of ethanol by alcoholic fermentation from starchy raw materials.

According to another preferred feature, the prefermentation nutrient medium is composed of or comprises a mixture of vinasses and liquefied wort, optionally water.

According to yet another preferred feature, the prefermentation nutrient medium is composed of or comprises a mixture of vinasses and nutritional supplement according to the invention, in particular fermented wheat bran, optionally water.

Finally, it is also possible to form a nutrient medium composed of or comprising all the abovementioned components.

The prefermentation nutrient medium according to the invention preferably contains a quantity of dry matter such that the stirring and/or aeration of the medium is sufficient to support an optimum ethanol production, for example of 15% to 20% of dry matter.

For the process for the manufacture of yeast according to the invention, the nutritional supplement according to the invention may be a fermented wheat bran. The vinasses derived from the fermentation or simultaneous saccharification and fermentation medium have a composition, in particular a content of sugars which can be used by the yeast under aerobic conditions, which is particularly favorable to the development of the yeast in the prefermenters. The prefermentation medium may be supplemented with exogenous nutritive components such as glycerol or glycerol solutions and with hydrolyzates obtained from moist brewers' grain. This advantageously makes it possible to save the glucose in the wort according to the invention and therefore to limit said reduction in yield.

Preferably, at least part of the vinasses is recycled by incorporation into the prefermentation nutrient medium. It is also preferable to recycle the vinasses after concentration in order to limit the dilution of the nutrient medium in the prefermenter.

Preferably, the vinasses present in the prefermentation nutrient medium completely replace the wort leaving the liquefaction, saccharification or presaccharification step. This replacement may however be only partial.

The addition of a nutritional supplement according to the invention, in particular of fermented wheat bran, also makes it possible to improve the prefermentation, in particular by enriching the nutrient medium with:
nitrogen, provided in the form of amino acids,
phosphorus,
sulfur,
essential vitamins and minerals,
proteins.

This addition may be made directly to the prefermentation nutrient medium or after mixing with wort leaving the liquefaction step, this mixture being added, after optional dilution, to the prefermenter.

The use of prefermented yeasts from the vinasses derived from a process for the manufacture of ethanol according to the invention also advantageously makes it possible to considerably enrich with proteins the dried brewers' grain obtained at the end of the process for the manufacture of ethanol according to the invention. Said brewers' grain may then contain more than 40% of proteins, and preferably at least 50% of proteins, on a dry matter basis.

The various enzymatic activities mentioned in the description and the claims were measured by the following methods:

Glucoamylase Activity.

The action of a glucoamylase (GA) preparation on a soluble starch solution causes the release of reducing sugars. When heated to 100° C. in the presence of 3,5-dinitrosalicylic acid (DNS), these compositions take on a brown color measured in a spectrophotometer (Kontron Instruments, Milan, Italy) at 540 nm.

The Reaction Medium Contains:

| | | |
|---|---|---|
| starch solution at 1.5% in the case | 1000 | μl |
| of Aspergillus niger and at 2% in | | |
| the case of Aspergillus orizae | | |
| citrate buffer 0.1 M at pH 4.5 | 900 | μl |
| enzyme solution: | 100 | μl |

The reaction runs for 20 minutes at 60° C. in the case of Aspergillus niger, for 5 minutes at 50° C. in the case of Aspergillus orizae. Samples of 100 μl of reaction medium are collected every 4 minutes in the case of Aspergillus niger and every minute in the case of Aspergillus orizae, mixed with 500 μl of DNS and 400 μl of citrate buffer at pH 4.5. The whole is then heated for 5 minutes at 100° C., rapidly cooled and then assayed at 540 nm against a blank consisting of a mixture of 500 μl of DNS and 500 μl of citrate buffer at pH 4.5.

These assay conditions were established after having studied the influence of the temperature and of the pH on the activity of the GA preparations. Soluble starch from Merck (Darmstadt, Germany) was used as substrate for this enzyme hydrolysis. The DNS is prepared according to the protocol proposed by P. Bernfeld, Methods in enzymology, 1, 149-158 (1955) which is the following:
Dissolve beforehand:
   10 g of 3,5-dinitrosalicylic acid,
   200 ml of sodium hydroxide 2 molars
   200 ml of distilled water.
Then add:
   300 g of double tartrate of sodium and potassium.
Adjust the volume to 1 liter with distilled water after complete dissolution.

Once prepared, this reagent should be stored protected from light. The calibration curves were established with glucose as reference product for the assay of the glucoamylase activity and for monitoring the liquefaction-saccharification reactions, and with xylose for measuring the xylanase activity.

One glucoamylase activity unit (GU) corresponds to the quantity of enzyme necessary to release one micromole of reducing ends per minute under the assay conditions with glucose as reference. The glucoamylase activity, calculated with the aid of the formula indicated below, is expressed in terms of the quantity of initial dry matter (IDM):

$$A = \frac{P \times {V}_{ferm}}{{V}_{enz} \times {M}_{ferm}}$$

A corresponds to the GA activity expressed as GU·gIDM⁻¹ (μmol·⁻¹·gIDM⁻¹)
P corresponds to the rate of release of glucose equivalents in μmol·min⁻¹,
V_{enz} represents the volume of enzyme solution assayed in ml,
V_{ferm} is the total volume of distilled water used to extract the enzyme solution in ml,
M_{ferm}, expressed in g of IDM, corresponds to the initial mass of dry product from which the enzyme solution was extracted.

Protease Activity.

This assay was developed on azocasein according to the method by Béinon, described in the book “Proteins Purification Methods—a Practical Approach”, Harris E.L.V. and Angal, S (Editors), IRL-Press, Oxford University Press, 1-66 (1989). The degradation of this substrate by proteases causes the release of azo groups which absorb in UV at 340 nm. The variation of the absorbance during the kinetics of hydrolysis of this protein indicates the extent of the reaction.

The Reaction Medium Contains:

| | | |
|---|---|---|
| azocasein solution at 1%, pH 5.0: | 1000 | μl |
| enzyme solution: | 200 | μl |

The azocasein (Sigma, Saint-Louis, United States) is dissolved in an acetate buffer 0.1 mol·l⁻¹ at pH 5.0. The protease activities were assayed at this pH because azocasein is insoluble in acetate buffer at lower pH values. The enzyme reaction is carried out at 60° C. Samples are collected every 5 minutes for 20 minutes and mixed with 5% trichloroacetic acid (TCA) in order to stop the reaction.

One protease activity unit (PU) corresponds to the quantity of enzymes necessary for an increase of 0.01 unit A₃₄₀ₙₘ per minute, generated by the release of azo groups under the conditions mentioned above. This activity, calculated from the formula indicated below, is expressed in terms of the initial dry matter (PU·gIDM⁻¹) or of the glucoamylase activity (PU·GU⁻¹):

$$A = \frac{P \times {V}_{ferm}}{{V}_{enz} \times {M}_{ferm}}$$

A corresponds to the protease activity expressed in PU·gIDM⁻¹,
P corresponds to the rate of release of the azo groups expressed as an increase of 0.01 unit A₃₄₀ₙₘ·min⁻¹,
V_{enz} represents the volume of the enzyme solution assayed in ml,
V_{ferm} is the total volume of distilled water used to extract the enzyme solution in ml,
M_{ferm} expressed in g of IDM, corresponds to the initial mass of dry product from which the enzyme solution was extracted.

Xylanase Activity

To demonstrate this enzymatic activity, the GA preparations were caused to act on a soluble xylan solution and the reducing sugars released were measured by the DNS method.

The Reaction Medium is Composed of:

| | | |
|---|---|---|
| xylan solution at 2%, pH 4.5: | 1900 | μl |
| enzyme solution: | 100 | μl |

The solution of larch xylan (Sigma at 1%) is prepared in 0.1 M citrate buffer at pH 4.5 and the reaction runs at 60° C. in the case of Aspergillus niger and at 50° C. in the case of Aspergillus orizae. Samples of 200 μl of reaction medium are collected every 2 minutes for 10 minutes, and mixed with 500 μl of DNS and 300 μl of citrate buffer at pH 4.5. The whole is then heated for 5 minutes at 100° C., rapidly cooled and then assayed at 540 nm against a blank consisting of a mixture of 500 μl of DNS and 500 μl of citrate buffer at pH 4.5.

One xylanase activity unit (XU) corresponds to the quantity of enzymes necessary for the release of one micromole of reducing sugars per minute. This activity is expressed in terms of the initial dry matter (XU·gIDM⁻¹) or in terms of the glucoamylase activity (XU·GU⁻¹). To calculate this activity, we again used the formula defined for the calculation of the GA activities in which:
A corresponds to the xylanase activity expressed in XU·gIDM⁻¹ (μmol·min⁻¹·gIDM⁻¹),
P corresponds to the rate of release of xylose equivalents in μmol·min⁻¹,
the other terms of the formula are not modified.

The following nonlimiting trials are given with the aim of illustrating the invention, in particular the advantage offered by the use of a fermented wheat bran according to the invention in the case of a simultaneous saccharification and fermentation process.

### EXAMPLE 1

### Effect of a Nitrogen Supplementation

The quantity of ethanol produced during a simultaneous saccharification and fermentation step was studied comparatively by introducing either a commercial enzyme preparation (Spirizyme® Fuel), or the same enzyme preparation with a nonlimiting nitrogen supplementation, or a nutritional supplement according to the invention.

In these trials, the inventors prepared 750 g of wheat flour wort containing 32% of dry matter in a stirred 1 liter reactor. After 2 h of liquefaction at 85° C. and pH 5.5, the wort is distributed into 3 Erlenmeyer flasks of 0.5 liter each containing 200 g of wort and adjusted to glucoamylase isoactivity as follows:
flask 1: Spirizyme® Fuel (purified glucoamylase solution, Novozymes);
flask 2: Spirizyme® Fuel and nitrogen supplementation, that is to say phosphate and diammonium sulfate: 3 g/l of each product;
flask 3: wheat bran fermented with an Aspergillus niger strain ATCC 76061, according to the process described in US 2002 037 342.

The initial pH of the worts in the three flasks was adjusted to 4.5 and the temperature to 32° C.

The incorporation of the nitrogenous products into flask 2 generates alkalinization of pH 6.2, requiring additional supply of concentrated hydrochloric acid in order to bring the initial pH of the wort to 4.5 for the simultaneous saccharification and fermentation. The worts are inoculated with 10⁷ CFU of yeast per ml of wort. The yeast used in the examples is Saccharomyces cerevisiae.

The maximum theoretical concentration of glucose in the wort is 303 WI after total enzymatic hydrolysis of the starch.

As shown in table 1, the best production of ethanol is obtained with the wheat bran fermented in 72 hours. The use of Spirizyme® Fuel, even with a nitrogenous supplement generates a maximum of only 59% of ethanol in 96 hours. In the absence of nitrogen incorporation, the production of ethanol is very low and a very high concentration of glucose is observed.

There is therefore a significant advantage in using wheat bran fermented by a mold for the production of ethanol from wheat even at the temperature of the simultaneous saccharification and fermentation reactor. The comparison with the Spirizyme Fuel indicates that a nonlimiting supplementation with nitrogen only partially explains the improvement in performance.

TABLE 1

| | Ethanol produced/ | | | Glucose produced/ | | |
|---|---|---|---|---|---|---|
| | final ethanol with the | | | final glucose with the | | |
| SSF | fermented bran (%) | | | fermented bran (%) | | |
| duration | | | Fermented | | | Fermented |
| (h) | Spirizyme | Spirizyme + N | bran | Spirizyme | Spirizyme + N | bran |
| 0 | 0.0 | 0.0 | 0.0 | 0.0 | 16.1 | 16.1 |
| 1 | 0.0 | 0.0 | 0.0 | 18.0 | 29.2 | 51.3 |
| 4 | 0.8 | 0.6 | 1.1 | 71.8 | 56.8 | 119.5 |
| 8 | 2.7 | 1.2 | 6.9 | 143.6 | 130.1 | 144.9 |
| 11 | 4.6 | 4.2 | 17.5 | 197.5 | 170.8 | 119.9 |
| 15 | 6.2 | 16.1 | 30.9 | 269.3 | 110.2 | 89.8 |
| 20 | 8.6 | 32.0 | 51.0 | 359.1 | 66.5 | 55.1 |
| 24 | 9.9 | 37.0 | 57.1 | 430.9 | 3.4 | 38.6 |
| 30 | 13.4 | 45.0 | 73.6 | 538.6 | 2.8 | 50.0 |
| 36 | 13.4 | 48.2 | 81.3 | 646.4 | 7.6 | 74.2 |
| 48 | 16.4 | 53.6 | 94.7 | 861.8 | 10.6 | 71.6 |
| 72 | 18.4 | 57.3 | 99.3 | 1292.7 | 133.9 | 81.8 |
| 96 | 16.9 | 59.0 | 100.0 | 1723.6 | 239.0 | 100.0 |

These trials confirm the suitability of the fermented wheat bran for the production of ethanol from wheat by a simultaneous saccharification and fermentation process.

### EXAMPLE 2

### Buffer Effect of the Nutritional Supplement According to the Invention

This example illustrates the advantages of the use of the nutritional supplement according to the invention for promoting simultaneous saccharification and alcoholic fermentation. This example relates in particular to the buffer effect of the nutritional supplement according to the invention on the simultaneous saccharification and fermentation medium.

In these trials, the inventors prepared 750 g of wort from wheat flour containing 32% of dry matter in a stirred 1 liter reactor. After 1 h 30 min of liquefaction at 85° C., at pH 5.5 and with stirring at 250 rpm, the wort is distributed into 2 Erlenmeyer flasks of 250 ml. Each flask contains 100 g of liquefied wort containing 32% of dry matter and 40 g of sterile water so as to obtain a wort containing 23% of dry matter. Next, the following are added to each wort:
flask 1: 0.5 g of wheat bran fermented with an Aspergillus niger strain ATCC 76061, according to the process described in US 2002 037 342;
flask 2: 16.7 μl of Spirizyme® Fuel (purified glucoamylase solution, Novozymes).

The initial pH of the worts in the two flasks is adjusted to 4. The worts are inoculated with 5×10⁶ CFU of yeasts Ethanol Red® (Lesaffre) per ml of wort.

Exogenous nitrogen, in the form of aqueous ammonia, is added to flask 2 in an amount of 1 gram of nitrogen per kilogram of wheat.

During the entire duration of the trials, the worts are maintained at a temperature of 30° C. and with stirring at 100 rpm.

The desired concentration of ethanol is 87 g of ethanol per liter of wort (11% v/v), which corresponds to a starch to ethanol conversion yield of 81%.

Table 2 shows the variation of the concentration of ethanol in the wort and the pH of the wort as a function of the duration of the simultaneous saccharification and fermentation.

In the same manner as in table 1, table 2 shows that the use of a nutritional supplement according to the invention in the simultaneous saccharification and fermentation medium makes it possible to obtain a higher concentration of ethanol in a shorter period than the conventional enzymes despite an exogenous nitrogen supply.

Furthermore, table 2 shows that the incorporation of ammonia into flask 2 causes a reduction in pH to around 2.8.

To obtain a better yield of ethanol, it is important that the pH of the wort remains between 3.5 and 5. Indeed, as the simultaneous saccharification and fermentation is carried out at about 30° C., an excessively high pH, in particular greater than 5, causes a risk of contamination of the wort. An excessively low pH value, in particular less than 3.5, causes a reduction in the yield of alcoholic fermentation.

The use of an enzyme preparation to which there is added an exogenous nitrogen source in a simultaneous saccharification and fermentation medium requires a device for regulating the pH, so as to maintain a pH value of between 3.5 and 4.5.

TABLE 2

| | Ethanol concentration (g/l) | | pH | |
|---|---|---|---|---|
| SSF duration | Fermented | Spirizyme + | Fermented | Spirizyme + |
| (h) | bran | NH₃ | bran | NH3 |
| 0 | 0 | 0 | 4 | 4 |
| 19 | 56.6 | 33.8 | 3.7 | 2.9 |
| 28 | 72.7 | 48 | | |
| 43 | 82 | 63.9 | 3.8 | 2.7 |
| 47 | 82.6 | 71 | | |
| 67 | 88 | 77.9 | 3.8 | 2.8 |

The nutritional supplement according to the invention has a buffer effect on the pH of the simultaneous saccharification and fermentation wort and therefore advantageously makes it possible to dispense with a device for regulating the pH.

### EXAMPLE 3

### Biostimulation

This example demonstrates the biostimulation effect of the nutritional supplement according to the invention. The trials of this example illustrate that the nitrogen supplementation and the effect of stabilization of the pH of the wort at around 4 only partially explain the improvement in the performance of the yeast for the production of ethanol during its introduction into the simultaneous saccharification and fermentation medium.

In these trials, the inventors prepared wort from wheat flour containing 32% of dry matter. After 1 h 30 min of liquefaction at 85° C., at pH 5.5 and with stirring at 250 rpm, the wort is distributed into 2 bioreactors of 4 liters. Each bioreactor contains 1.7 kg of wort containing 29.8% of dry matter. The wort obtained in each bioreactor is obtained by mixing the wort containing 32% of dry matter derived from the liquefaction with vinasses containing 27% of dry matter.

The vinasses used to dilute the wort are vinasses obtained at the end of a process for the alcoholic fermentation of wheat.

The 1.7 kg of wort containing 29.8% of dry matter are supplemented as follows:
bioreactor 1: 199 μl of Spirizyme® Fuel (purified glucoamylase solution, Novozymes) and 59 μl of Viscozym Wheat® (Novozyme) (deviscosifying enzyme);
bioreactor 2: 5.7 g of wheat bran fermented by an Aspergillus niger strain ATCC 76061, according to the process described in US 2002 037 342.

The initial pH of the worts in the two bioreactors was adjusted to 4.1 and the temperature to 30° C., and then the worts are subjected to stirring at 100 rpm.

The worts are then inoculated with 5×10⁶ CFU of yeasts (Ethanol Red®, Lesaffre), per ml of wort.

Exogenous nitrogen is added to bioreactor 1, in fractions, in the form of aqueous ammonia in order to arrive at a content of 1 gram of nitrogen per kilogram of wheat.

The vinasses derived from wort from fermented cereals contain a certain quantity of fibers. By mixing them with the wort, a buffer effect of the pH of the wort is obtained.

The desired concentration of ethanol is 87 g of ethanol per liter of wort, which corresponds to a starch to ethanol conversion yield of 81%.

Table 3 shows the variation of the concentration of ethanol in the wort and the pH of the wort as a function of the duration of the simultaneous saccharification and fermentation.

The fractionation of the exogenous nitrogen supply and the use of vinasses allows stabilization of the pH in the bioreactor 1 at around 4.

As shown in table 3, the desired concentration of ethanol is almost reached in 28 hours in the bioreactor with a nutritional supplement according to the invention whereas 44 hours are required to reach an equivalent concentration in the bioreactor with the enzyme preparation. Furthermore, after 68 hours, the ethanol concentration is still higher in the bioreactor with the nutritional supplement according to the invention than in that with the enzyme preparation.

Table 3 illustrates the significant advantage of the use of a nutritional supplement according to the invention compared with conventional enzyme preparations. The results of these trials indicate that a nitrogen supplementation and a pH control only partially explain the performance of the nutritional supplement according to the invention.

These trials demonstrate a biostimulation effect of the yeasts linked to the composition of the nutritional supplement according to the invention.

TABLE 3

| | Ethanol concentration (g/l) | | pH | |
|---|---|---|---|---|
| SSF duration | Fermented | Spirizyme + | Fermented | Spirizyme + |
| (h) | bran | NH₃ + VW | bran | NH₃ + VW |
| 0 | 0 | 0 | 4.1 | 4.1 |
| 8 | 6.8 | <4 | 4 | 4.1 |
| 20 | 69 | 35.5 | 3.9 | 3.75 |
| 24 | 73.5 | 48.8 | 4 | 3.8 |
| 28 | 82.5 | 58.5 | 4 | 3.9 |
| 44 | 85.6 | 82.6 | 4.1 | 3.8 |
| 48 | 93.6 | 88.9 | 4.1 | 3.85 |
| 68 | 97.1 | 91.3 | 4.2 | 4 |

### EXAMPLE 4

### Aerobic Fermentation of the Vinasses

The inventors carried out a comparative study of the variation of the yeast concentration of the prefermentation medium as a function of the dry matter content of said medium.

These trials were carried out on 100 g of prefermentation medium in 250 ml baffled flasks. The prefermentation media are thermostatted at 30° C., placed under stirring at 130 rpm and inoculated with 2.5×10⁷ yeasts (Ethanol Rede), per ml of medium. The initial pH of the media is 4.2 to 4.4

Each prefermentation medium is composed as follows, as a percentage by mass:
flask 1: 1.2% of vinasse containing 28% of dry matter, and about 98.8% of water;
flask 2: 10% of vinasse containing 28% of dry matter, and about 90% of water;
flask 3: 20% of vinasse containing 28% of dry matter, and about 80% of water;
flask 4: 40% of vinasse containing 28% of dry matter, and about 60% of water;
flask 5: control medium (glucose and yeast extract).

The flasks are stirred so as to oxygenate the medium and allow propagation of the yeast.

The inventors measured the variation of the concentration of yeast in each prefermentation medium.

The compositions of the media in the flasks and the variations of the yeast concentrations are summarized in the following table 4:

TABLE 4

| | Count (yeasts/ml) | | | | |
|---|---|---|---|---|---|
| Duration of | 1.2% | 10% | 20% | 40% | |
| culture (h) | vinasses | vinasses | vinasses | vinasses | Control |
| 0 | 2.50E+07 | 2.50E+07 | 2.50E+07 | 2.50E+07 | 2.50E+07 |
| 5 | 3.30E+07 | 4.90E+07 | 1.54E+08 | 4.15E+08 | 4.90E+07 |
| 7 | 3.00E+07 | 2.50E+08 | 3.10E+08 | 4.50E+08 | 6.00E+07 |
| 24 | 3.30E+07 | 1.90E+08 | 3.50E+08 | 5.50E+08 | 2.00E+08 |

As shown in table 4, a prefermentation medium composed at 40% by mass of vinasse containing 28% by mass of dry matter, that is a medium containing 11.2% by mass of dry matter, allows multiplication by a factor of 16.6 of the quantity of yeast in 5 hours. By way of comparison, the multiplication factor over the same period in the control medium is about 2.

After 24 hours of aerobic culture, the concentration of yeast in the medium composed of 40% by mass of vinasse is more than 2.5 times the concentration of yeast in the control medium.

Without being bound by a theory, the inventors explain these results by the presence in the dry matter of the vinasses of carbon sources, other than glucose, which are assimilable by the yeast. The inventors have shown that the dry matter of the vinasses contains about 10% of fermentable sugars, mainly in the form of glucose or residual starch. The initial concentration of yeast corresponds to about 0.62 g of yeast per liter of medium, while the final concentration corresponds to about 13.8 g of yeast per liter of medium. About 13.2 g of yeast per liter of medium were therefore formed, which corresponds to a consumption of about 26.4 g of glucose. Now in flask No. 4, the initial concentration of glucose is about 11.2 g per liter of medium. The quantity of yeast obtained is therefore linked to another source of carbon than glucose, for example the presence of glycerol in the vinasses.

Of course, the present invention is not limited to the embodiments described and represented which are provided by way of illustrative and nonlimiting examples.

1. Nutritional supplement for alcoholic fermentation medium from carbohydrate raw materials, comprising an active ingredient, in crude or extracted form, derived from a fermentation with a mold.

2. Nutritional supplement according to claim 1, in which the carbohydrate raw material is a starchy raw material.

3. Nutritional supplement according to claim 1, comprising a wheat bran fermented with a mold.

4. Nutritional supplement according to claim 1, having at least one of the following enzymatic activities:
glucoamylase: at least 500 GU, per gram of dry matter,
proteolytic: at least 100 PU per gram of dry matter,
xylanase: at least 100 XU per gram of dry matter.

5. Nutritional supplement according claim 1, in which the active ingredient is chosen from the group consisting of ergosterol, N-acetylglucosamine, vitamins, nucleic acids, amino acids and mixtures thereof.

6. Nutritional supplement according to claim 1, obtained by fermentation of a wheat bran with a mold chosen from the Aspergillus niger strains ATCC 201202, ATCC 76060, ATCC 76061, MUCL 28815, MUCL 28816, NRRL 3112 or from the Aspergillus orizae strains ATCC 22788 and ATCC 42149.

7. Nutritional supplement according to claim 1, which can be obtained by means of a process comprising the following steps:
i) taking wheat bran,
ii) moistening, acidifying to a pH of between 4 and 5 and heat treating said bran so as to pasteurize it or sterilize it,
iii) inoculating the resulting wheat bran with an Aspergillus niger strain, chosen from ATCC 201202, ATCC 76060, ATCC 76061, MUCL 28815, MUCL 28816, NRRL 3112, or with an Aspergillus orizae strain chosen from ATCC 22788 and ATCC 42149,
iv) fermenting the wheat bran in the solid state in a reactor which is periodically stirred, at a temperature of 28° C. to 38° C., the moisture content being maintained between 45% and 65% by weight, under aeration conditions designed to avoid a permanent accumulation of carbon dioxide in the wheat bran until the fermentation product has the following minimum enzyme activity values:
glucoamylase: at least 500 GU per gram of dry matter and, optionally,
proteolytic: at least 100 PU per gram of dry matter,
xylanase: at least 100 XU per gram of dry matter.

8. Nutritional supplement according to claim 7, the heat treatment in step ii) being preferably subsequent to the moistening.

9. Nutritional supplement according to claim 1, in which the glucoamylase activity is at least 750 GU per gram of dry matter.

10. Nutritional supplement according to the preceding claim, in which the glucoamylase activity is at least 1500 GU per gram of dry matter.

11. Nutritional supplement according to claim 1, comprising ergosterol, N-acetylglucosamine, vitamins, nucleic acids and amino acids.

12. Nutritional supplement according to claim 1, comprising vitamin B.

13. Nutritional supplement according to claim 1, comprising one or more amino acids chosen from: alanine, arginine, asparagine, aspartic acid, valine, leucine and glutamic acid.

14. Process for the manufacture of ethanol from a carbohydrate material, comprising a step of fermentation in the presence of a nutritional supplement according to claim 1.

15. Process for the manufacture of ethanol according to claim 14, in which the carbohydrate material is a starchy material, comprising, after a liquefaction step and optionally a presaccharification step, a simultaneous saccharification and fermentation step in a simultaneous saccharification and fermentation medium whose glucose content at the start of the simultaneous saccharification and fermentation step is at most equal to 95% of the glucose equivalent corresponding to the starch of said starchy material, characterized in that the nutritional supplement is introduced into the simultaneous saccharification and fermentation medium.

16. Process according to claim 15, in which the glucose content at the start of the simultaneous saccharification and fermentation step is at most equal to 3% of the glucose equivalent corresponding to the starch of said starchy material.

17. Process according to claim 15, the nutritional supplement being introduced in crude form, in which the quantity of said nutritional supplement is greater than or equal to 4 kg of dry matter and/or less than or equal to 60 kg of dry matter per ton of starch.

18. Process according to claim 15, in which said nutritional supplement constitutes the main nutritional source for the yeast of the simultaneous saccharification and fermentation medium and/or the main source of ergosterol and/or the main source of nitrogen and/or of amino acids and/or of phosphorus and/or of sulfur and/or of vitamins.

19. Process according to claim 15, in which the simultaneous saccharification and fermentation is entirely carried out in the absence of an air or oxygen supply.

20. Process according to claim 15, in which the simultaneous saccharification and fermentation comprises an initial aerobic phase.

21. Process according to claim 15, in which the simultaneous saccharification and fermentation is performed under the following conditions:
temperature: from 30° C. to 35° C.,
pH: adjusted at the start of the simultaneous saccharification and fermentation step to between about 3.5 and about 5,
yeast inoculum: about 10⁶ to about 5×10⁸ CFU of yeast per ml of simultaneous saccharification and fermentation medium,
20% to 35% of DM,
residence time: 20 hours to 72 hours.

22. Brewers' grain derived from a process for the manufacture of ethanol according to claim 14, containing more than 5 g of ergosterol per ton of dry matter of brewers' grain.

23. Brewers' grain according to claim 22, containing more than 40%, preferably more than 50% of crude proteins, as percentages on the basis of the dry matter.

24. Brewers' grain according to claim 22, containing more than 50% of crude proteins, as percentages on the basis of the dry matter.

25. Use of a nutritional supplement according to claim 1, for promoting an alcoholic fermentation or a prefermentation intended for the manufacture of a yeast.

26. Process for the manufacture of yeast under aerobic conditions in a prefermenter, characterized in that there are added to said prefermenter clarified vinasses and/or concentrated vinasses obtained during the carrying out of a process for the manufacture of ethanol by alcoholic fermentation from carbohydrate raw materials.

27. Process for the manufacture of yeast under aerobic conditions in a prefermenter, characterized in that there are added to said prefermenter clarified vinasses and/or concentrated vinasses obtained during the carrying out of a process for the manufacture of ethanol by alcoholic fermentation from carbohydrate raw materials wherein the vinasses are obtained during the carrying out of a process for the manufacture of ethanol into which has been incorporated a nutritional supplement according to claim 1.

28. Process, for the manufacture of yeast under aerobic conditions in a prefermenter, characterized in that there are added to said prefermenter clarified vinasses and/or concentrated vinasses obtained during the carrying out of a process for the manufacture of ethanol by alcoholic fermentation from carbohydrate raw materials wherein the vinasses are obtained during the carrying out of a process according to claim 14.

29. Process for the manufacture of ethanol from a carbohydrate material, comprising a step of fermentation in the presence of a nutritional supplement including an active ingredient, in crude or extracted form, derived from a fermentation of a mold, in which yeasts obtained by a process for the manufacture of yeast according to claim 26 are used for the ethanol fermentation, which process uses vinasses obtained from said process for the manufacture of ethanol.
